(19) United States
(12) Patent Application Publication
(10) Pub. No.: US 2005/0209546 A1
(43) Pub. Date: Sep. 22, 2005

(54) Remote control for extracorporeal blood processing machines

(75) Inventors: Jorgen Jonsson, Sjobo (SE); Allan Petersen, Bjarred (SE)

Correspondence Address: GAMBRO, INC;PATENT DEPARTMENT, 10810 W COLLINS AVE, LAKEWOOD, CO, 80215, US

(73) Assignee: Gambro Lundia AB, Lund (SE)

(21) Appl. No.: 10/903,232

(22) Filed: Jul. 29, 2004

Related U.S. Application Data
(63) Continuation of application No. 09/736,072, filed on Dec. 13, 2000, now abandoned.

(30) Foreign Application Priority Data
Dec. 22, 1999 (SE) 9904782-1

(51) Int. Cl. A61M 37/00

(52) U.S. Cl. 604/4.01

(57) ABSTRACT

A remote control sub-system for use in controlling a fluid circuit wherein the fluid circuit includes at least one tubing segment which is operatively engaged with a flow control device such as a pump so that the pump is operable to force fluid to flow in and through the tubing segment. The remote control sub-system has a remote control device with a switching member operatively connected thereto; the remote control device being disjoined from the flow control device yet being disposed in operative communication therewith such that activation of the switching member is effective to turn on or off the flow control device to start or stop the flow of fluid through the tubing segment.

The present application is a Continuation of application Ser. No. 09/736,072, filed on Dec. 13, 2000, which claims foreign priority benefits under 35 U.S.C. 119 from Swedish application number 9904782-1 filed on Dec. 22, 1999.

### BACKGROUND OF INVENTION

The present invention is directed generally to remote control devices and more particularly to such devices as used in the operation and control of extracorporeal blood processing machines.

Extracorporeal blood processing systems generally involve the removal of blood from a patient's body, flowing it to and through a blood processing apparatus and then usually returning it to the patient. The blood is most often drawn from the patient through a blood removal needle, cannula or like device inserted into a patient's vein or artery and then returned to the body through a return needle, cannula or like device. A circuit of tubing segments provides for the blood flow to the processing apparatus or apparatuses and then back to the patient.

Insertion and extraction of the blood removal and return needles or like devices are particularly problematical in extracorporeal blood processing. For example, in an extracorporeal procedure generally known as dialysis, a patient is often subjected to treatment three or more times per week. As is understood in the art, great care must be taken during needle insertion and extrication to ensure the continued viability of a patient's access site, where a needle is inserted into the patient's vasculature, vein or artery. Care is particularly crucial in dialysis and like procedures because of the high number of instances of repeated vascular puncturing for blood removal and return. Improper or careless needle handling during needle insertion or removal can cause serious damage to the access vasculature, potentially rendering such a site inaccessible for future use.

Conventional extracorporeal processing machinery and disposable tubing sets have been developed to incorporate numerous enhanced user features. User customization of operation variables is one area of common advance, although higher degrees of automation and operator ease are also being developed. Nevertheless, these general areas of development are often contradictory to each other because increasing user choice in customization and variable control often counters or reduces the level of automatic control the machine would then perform. Moreover, certain functions continue to preferably be subject to human judgment and consequent input, and thus remain outside the range of purely automated machine operation.

Two such areas of extracorporeal machine operation preferably remaining in the control of qualified practitioners involve initiating blood flow at the beginning of a procedure and shutting the machine down at the completion of a procedure. At the beginning of a procedure, the blood removal needle is inserted into a patient's vascular access site and then the pump is started to initiate blood flow from the patient through the tubing circuit. Conventionally, the practitioner's attention is drawn, even if only temporarily, from the patient to the machine in order to start the pump. Similarly, at the end of a conventional treatment, the removal needle is taken out of the patient's access site by the practitioner and is then connected to a source of saline solution such as a saline bag. This step also requires the drawing of attention away from the patient. The pump may be continually moving or more preferably is manually stopped and then restarted after connection to the saline source. Again, the practitioner must at least temporarily go to the machine to stop and restart the pump. The blood in the tubing is then pumped through the system with saline solution following therebehind until all of the blood is forced through the return needle back into the patient. When only saline solution is left in the tubing system, the practitioner then stops the flow of fluid, usually by going to the machine and stopping the pump. The blood return needle may then be removed by the practitioner.

As mentioned, these various conventional steps require practitioner attention to numerous diverse activities occurring simultaneously, or nearly simultaneously, with the ever important insertion and extrication of the blood removal and return needles.

Needle insertion is closely followed by pump starting and consequent attention to the adequacy of blood flow. The extrication of the removal needle is followed closely by its insertion into a saline bag or like fluid -source. The practitioner is usually also concerned with stopping and restarting the blood pump during this finishing phase. Soon thereafter, the removal of the return needle is coincident closely in time with the ultimate stopping of the fluid pump by the practitioner.

This direction of a practitioner's concentration to so many simultaneous and/or consecutive tasks presents a distinct problem primarily in the drawing of attention away from the patient's vascular access blood removal and return sites. As described, great care must be taken in the handling of these sites to prevent damage thereto. During and after removal of the needles, manual pressure must be consistently applied to these sites to arrest bleeding and achieve hemostasis, thereby promoting natural closure and healing of the puncture opening. The drawing of the practitioner's attention away from these sites for pump control, inter alia, decreases practitioner attention to providing proper hemostatic pressure and thereby increases the risk of vascular damage. Thus, there exists a significant need to reduce the practitioner's distraction from the vascular access sites during extracorporeal initiation and completion procedures.

Similar also is the problem of practitioner distraction during an episode of patient hypotension during dialysis. If a patient undergoing dialysis loses too much fluid, that patient will likely experience hypotension or a sudden drop of blood pressure often accompanied by nausea, vomiting and potential fainting. The patient is obviously then in need of nursing care for these symptoms, but also particularly in need of an infusion of a saline solution to increase the blood volume. However, as above, practitioner attention to the mechanics of beginning a saline infusion usually necessarily requires disattention to the patient and treatment of the patient's immediate symptoms of hypotension. Thus, the provision of an automated infusion solution to limit practitioner distraction is sorely wanted. A disparate prior attempt involving the delivery of sodium to the dialysate is described in the U.S. patent to Keshaviah et al., U.S. Pat. No. 5,346,472; and the corresponding EP 0652780 B1 to Baxter Inc.

### SUMMARY OF INVENTION

The present invention is directed to providing a remote control device for an operator to initiate and/or stop extracorporeal machine functions without having to divert attention away from the patient.

In a simple form, the remote control involves a device which is generally disjoined from the extracorporeal processing unit and yet communicates therewith to control the stopping and/or starting of the fluid pump. Particularly useful is such a device which can be operated from the patient's side adjacent the vascular access blood removal and/or return sites. Similarly, a device which may be foot operated or otherwise activated without necessitating use of a practitioner's hand or hands is contemplated herein. Voice activation is also an alternative.

Moreover, additional functional devices and/or method steps may be added to simplify the procedure of completing an extracorporeal process. For example, a saline solution source may be interconnected to the tubing system in a manner that eliminates the need for using the blood removal needle as the connector to the saline source. The remote control of the present invention may then include distinct or inherent controls for opening and/or closing the saline interconnection to initiate or halt the flow of saline solution into the fluid circuit. A removal line clamp may similarly be subject to remote control. These remote control features may be automated by a single button or have separate manual means on the remote device.

The steps to be taken range from simply having the practitioner, who in focusing attention toward the vascular access blood removal and/or return sites, extract the blood removal needle while using the remote control device to stop and/or restart the pump. Also, the remote control may be used for the finishing process such that the practitioner watches until all the blood is run through the extracorporeal processing system, then stops the fluid pump using the remote control device, and then removes the return needle from the patient. A single needle system would only entail the remote starting and stoppage of the pump at appropriate times and then ultimately the removal of the single needle at the appropriate time at the end of the extracorporeal procedure.

Accordingly, a primary object of the present invention is the provision of remote control operation of extracorporeal processing devices to limit distraction of a practitioner's attention away from a patient.

These and other objects, features and advantages of the present invention will be further apparent by reference to the following detailed description read in conjunction with the accompanying drawings which are described briefly below.

### BRIEF DESCRIPTION OF DRAWINGS

In the drawings:

FIG. 1 is an isometric view of an extracorporeal fluid processing system using a remote control sub-system according to the present invention.

FIG. 1A is an enlarged, partial view of a portion of the remote control sub-system of FIG. 1 while in use.

FIG. 2 is an enlarged, rotated view of a portion of the remote control sub-system of FIG. 1.

FIG. 3 is a schematic view of the extracorporeal fluid circuit of the processing system of FIG. 1.

FIG. 4A is a block diagram which depicts a discrete set of process steps associated with using a remote control sub-system according to the present invention.

FIG. 4B is a block diagram which depicts another discrete set of process steps associated with using a remote control sub-system according to the present invention.

FIG. 5 is an alternative schematic diagram using a remote control sub-system according to the present invention.

FIG. 6 is a block diagram depicting process steps associated with using a remote control sub-system according to the present invention preferably with a processing system such as that shown in FIG. 5.

FIG. 7 is yet another alternative schematic diagram using a remote control sub-system according to the present invention.

### DETAILED DESCRIPTION

The present invention is directed primarily to providing practitioners a higher degree of simplification in controlling extracorporeal blood processing machines particularly while a practitioner's attention is necessarily directed toward the patient and the patient's vascular access blood removal and/or return sites.

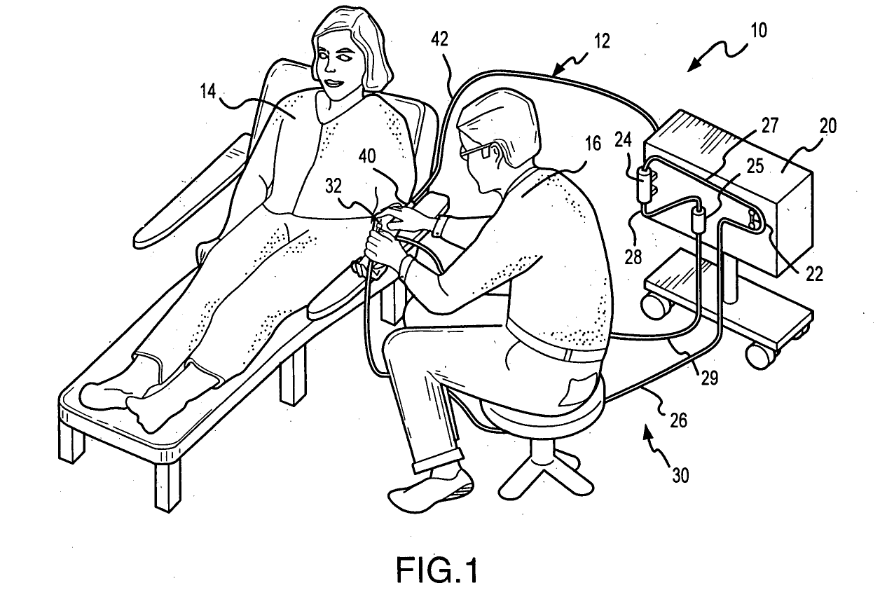

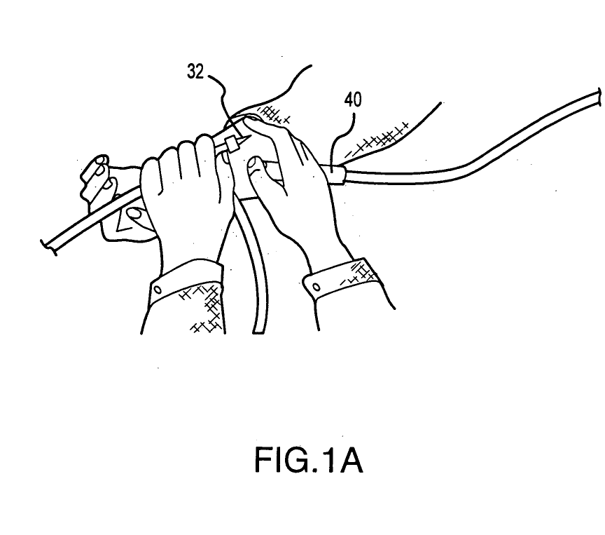

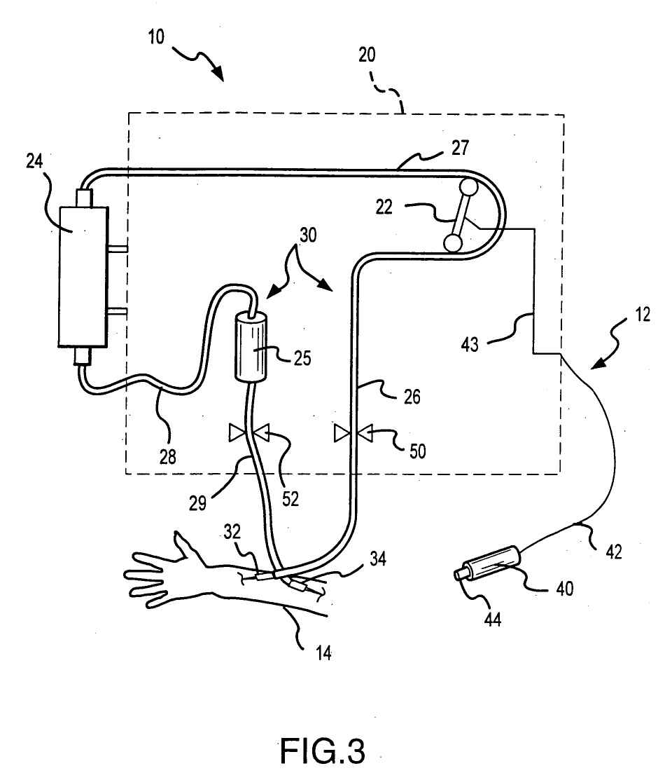

An extracorporeal blood processing system 10 incorporating a remote control sub-system 12 according to the present invention is shown in FIG. 1 of the attached drawings. FIG. 1 shows the extracorporeal system 10 and the remote control sub-system 12 in use on a patient 14 as controlled by a practitioner 16. The extracorporeal system 10 generally includes a control unit 20 which has a plurality of fluid flow control, monitoring and/or processing devices disposed thereon as is understood in the art. For example, unit 20 preferably includes at least a pump 22, a processing device 24 and an air or gas bubble trapping or detecting device 25. As shown, pump 22 is peristaltic, but it may alternatively be centrifugal or of another known pump type. Also, processing device 24 may be a semi-permeable filtration device also known as a dialyzer (flat-plate, hollow fiber, etc.) as shown, or another known processing device such as a centrifuge or an adsorption column (neither of which being shown in FIG. 1). Still further, air bubble device 25 may be a bubble trap as shown or a bubble sensor or both as is known in the art. One or more fluid tubing members, here shown including tubing members 26, 27, 28 and 29, are operatively connected to or associated with these process devices to create an extracorporeal fluid circuit 30. Fluid circuit 30, including its primary operative components among other contributing parts are shown and described in more detail relative to FIG. 3, below.

Connected to the relative free ends of tubing segments 26 and 29 distal from the control unit 20 are respective blood removal and return devices 32 and 34 (device 34 being hidden in FIG. 1, but is shown in FIG. 3, see below). These devices are inserted in the patient 14 for, respectively, removal of blood from and return of blood to the patient 14. Devices 32 and 34 are, as is known in the art, needles, catheters, cannulas and/or like devices which are insertable in the patient's access site vasculature; veins or arteries. Devices 32 and 34 and/or a single needle device (not shown) may thus also each be referred to as access devices. FIGS. 1 and 1A show the practitioner 16 either in the process of inserting or perhaps more appropriately readying to extricate the removal device 32 from the vascular access site of the patient 14. Note, the relative orientation of device 32 as inserted in the patient 14 may be as shown in FIG. 1, or as may be appropriate under certain circumstances as understood in the art, it may be disposed in an opposite orientation as shown in more detail in FIGS. 3, 5 and 7, below.

FIGS. 1 and 1A also show, at least partially, a few elements of remote control sub-system 12; namely, a hand-held remote control device 40 (in the practitioner's right hand) which is shown connected via an elongated cable 42 to the control unit 20 of the extracorporeal blood processing system 10.

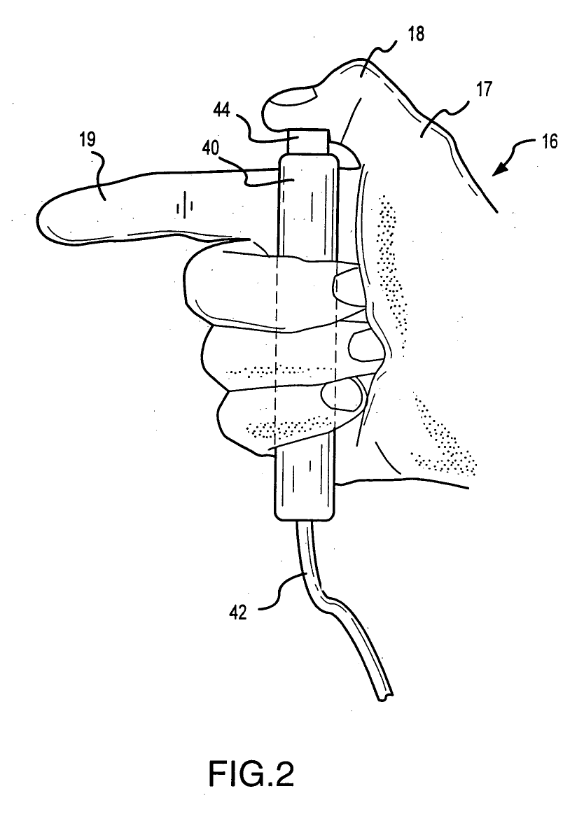

FIG. 2 shows a more detailed view of a hand-held remote control device 40 as gripped by the practitioner 16. The grip in FIG. 2 is the same as that shown in FIGS. 1 and 1A, but the view is from below, so that device 40 may be seen in more detail. In particular in FIG. 2, the hand 17 of the practitioner 16 is shown with the thumb 18 disposed adjacent a thumb-engaging push-button 44. In this embodiment, button 44 is a push-button toggle type of switching member which is primarily useful for switching the pump 22 either on or off as desired. Other embodiments of hand-held and non-hand-held devices are foreseeable within the present invention as will be described below. The practitioner's forefinger 19 is shown extended for applying hemostatic pressure to the vascular access site as shown in FIGS. 1 and 1A and as described in more detail below.

FIG. 3 is a schematic diagram of the extracorporeal system 10 of FIG. 1. In particular, the FIG. 3 schematic shows a plurality of tubing segments 26, 27, 28 and 29 which form the majority of the fluid circuit 30. Control unit 20 is shown in dashed lines to provide greater emphasis to the circuit 30. Also included in circuit 30 is the processing apparatus 24 through which the blood flows and is processed before returning to the patient 14. The pump 22 and the air bubble device 25 as introduced above are also schematically depicted in FIG. 3. As shown, the respective tubing segments 26 and 27 represent portions of a continuous member passing through pump 22, although it is known that they may instead be separately manufactured segments mechanically joined either to each other or to respective pump inlet and outlet couplings (not shown) as may be necessary or desired for a particular pump. Similarly, the respective tubing segments 28 and 29 are shown as separately attached to discrete ends of the air bubble device 25; however, it is also known that air bubble device 25 may simply be a sensor through which a continuous tubing segment 28/29 may be disposed.

The hand-held remote control device 40 is also shown in FIG. 3 as schematically connected to pump 22 to toggle pump 22 on and off during operation. This schematic connection is shown via the external cable 42 connected to an internal electrical line 43 disposed within control unit 20. Various internal electrical components (not shown) may be used in the actual electrical connection between the remote control 40 and the pump 22 as would be understood in the art.

Briefly, blood flow through fluid circuit 30 is as follows. Blood is removed from the patient 14 via blood removal needle 32 and flows through tubing segment 26 to pump 22 which forces the blood through tubing segment 27 to and through processing apparatus 24. Processed blood exits the processing apparatus 24 and flows through tubing segment 28 to the air bubble device 25 where air is either removed or detected or both. Finally, the blood flows from the air bubble device 25 to the patient 14 via tubing segment 29 and enters the patient 14 by flowing through the blood return device 34 inserted in the patient's vascular system.

Note, the fluid circuit 30 and extracorporeal system 10 described throughout this specification are generalized for facility in description of the present invention.

Other functions and features as are known in the art may also be incorporated herein without undue impact on the functionality of the present invention. For example, anticoagulant, medicament and/or saline tubing circuit connections/additions (not shown) may be included as desired. Also, pressure and other sensors_(not shown) may be used. These elements and their functionality, though having not been shown, are understood in the art. Nevertheless, two further control devices 50 and 52 are shown schematically in FIG. 3. These are relatively conventional control unit-operated clamping devices for stopping the flow of fluids in tubing segments 26 and 29, respectively. Clamping devices 50 and 52 are disposed on and operated by control unit 20, but may also be made subject to control by the remote control device 40 as will be described below. Though clamps are shown and described herein, valves or other flow control devices may similarly or alternatively be substituted herefor without altering the spirit or scope of the present invention.

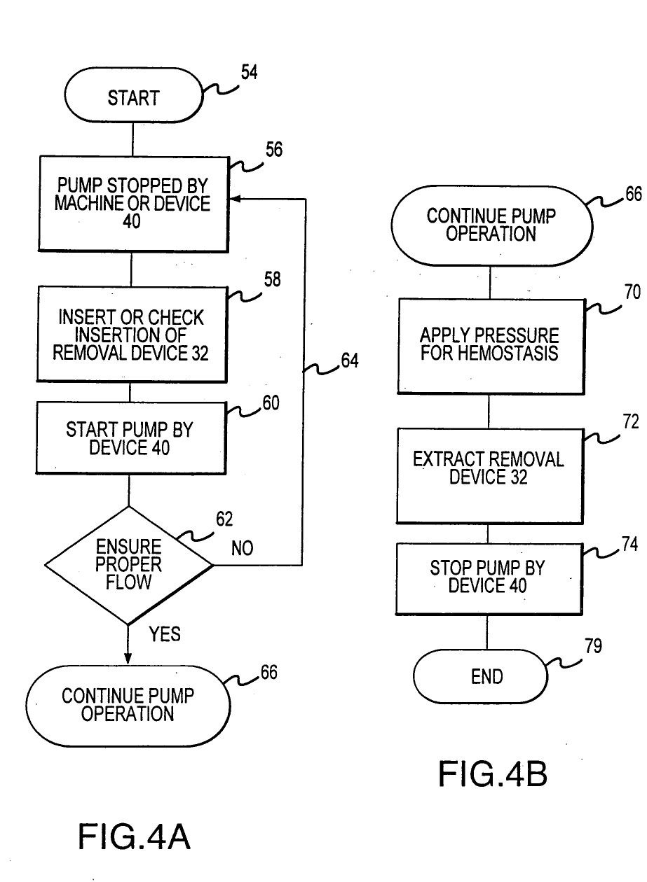

In operation according to the embodiment described thusfar relative to FIGS. 1-3, the practitioner 16 may use the remote control device 40 during initial blood flow starting procedures as well as during any stopping of blood flow. FIGS. 4A and 4B illustrate the steps taken for each such procedure. First, as shown in FIG. 4A, the process is begun at the ‘start’ oval 54, at which point it is presumed that all other conventional initial procedures have been performed, as for example, the connecting of the preferably disposable tubing circuit 30 and the processing apparatus 24 in operative position relative to the control unit 20, and the priming of the fluid circuit 30 with saline solution as is known in the art. Note, priming may be performed automatically or manually as in conventional machines, or control device 40 may also be used during priming for starting and/or stopping the pump 22 to introduce priming solution into the circuit 30; see below. Then per the first process box 56 in FIG. 4A, the pump 22 is confirmed to be stopped, either automatically by the machine 20, or manually by a practitioner engaging either the remote control device 40 or a manual switch on the machine 20, and the practitioner 16 is then ready to insert the blood removal device 32 into the vascular system of the patient 14 per the second process box 58. In particular, the practitioner 16 may then hold the control device 40 in one hand as shown in FIGS. 1, 1A and 2, for example, and carefully insert the blood removal device 32 into operative position within the vascular access site of the patient 14. The practitioner 16 may then, as depicted by the next process box 60, start the pump 22 using the remote control device 40 without removing attention from the vascular access site where the blood removal device 32 is inserted in patient 14. Depression of button 44 starts the pump 22.

Next, per the decision diamond 62 shown in FIG. 4A, the practitioner 16 ensures (visually or otherwise) that proper flow is obtained through blood removal device 32, into tubing segment 26 and into and through the rest of the fluid circuit 30. If proper flow has not been reached, then the practitioner may simply push the button 44 on the hand-held remote control device 40 to stop the pump 22 and then manually and/or visually check the insertion of the blood removal device 32. Re-insertion may be necessary. This process loop-back is shown in FIG. 4A by the flow path line 64 which takes the process back to boxes 56 and 58, stopping the pump and checking the insertion of the blood removal needle 32. If, on the other hand, proper flow has been obtained, then the process moves to continued pump operation as indicated by the process oval labeled 66 in FIG. 4A. This continued pump operation is then maintained for the duration of the extracorporeal processing session until a therapeutic goal has been obtained.

Insertion of blood return device 34 would then follow the same or a similar procedure at an appropriate time as understood in the art. The enhancement resides in the ability to ensure pump 22 stoppage and then the re-starting of pump 22 through use of device 40 without removing attention from the patient 14 at any time. Thus, attention can be maintained on the patient 14 from the insertion of removal device 32 through the insertion of the return device 34 and full ensurance of proper blood flow in the fluid circuit 30 from the patient and back.

Next, as shown in FIG. 4B, the continued pump operation oval 66 appears as the initial process point in a second sub-procedure in which the remote control device 40 may be used. In particular, at the end of an extracorporeal processing operation, the practitioner 16 may hold the remote control device 40 in one hand just as in the start procedure described above relative to FIG. 4A. The practitioner 16 may then use his or her other hand to extract the blood removal needle 32 from the patient's access site. Preferably simultaneously with the extrication of the removal needle 32, the practitioner uses at least one digit (such as a finger 19; see FIGS. 1, 1A and 2) to apply pressure to the access site and thereby provide for the achievement of hemostasis. These two steps are identified by separate process boxes 70 and 72 in FIG. 4B. Note, these steps may be interchanged such that blood removal device extrication occurs either before or subsequent to, or, as described above, simultaneously with pressure application for hemostasis. Also preferably simultaneously or very nearly so, the practitioner 16 would be able to engage the toggle switch 44 of the remote control device 40 to stop the pump 22. This is depicted in FIG. 4B by process box 74. Alternatively, pump stoppage may also have been the first step, prior to application of hemostatic pressure and extrication of the removal needle 32. As shown in FIGS. 1-3, the hand-held remote control device 40 provides a simple ability to perform these three steps either simultaneously or consecutively, or in any desirable order without distracting attention from the patient. Then, the remote control assisted procedures are either at completion, as signified by the end oval 79 in FIG. 4B, or finishing procedures may then be conducted as generally known in the art, by, for, example, inserting the now extricated blood removal device 32 into a saline solution source or bag (not shown in FIGS. 1-4). Then the pump 22 may be restarted, and according to the present invention this restarting may preferably be by simple depression of the toggle button 44 on remote control device 40 to reinitiate flow through the fluid circuit 30. Saline would then be pumped into the fluid circuit following behind any blood left therein. This phase of pump operation may then be continued until all of the blood is pumped out of the fluid circuit 30 back into the patient 14. Moreover, the practitioner may still be engaged with applying pressure to the patient's access site throughout all or most of this alternative procedure as well.

Note also that flow control clamps 50 and 52 may also be remotely manipulated into either the appropriate open or closed positions during these sub-procedures. Similarly, pump speed may alternatively or additionally be controlled by remote control device 40 during any of these stages of operation such as during ensurance of proper flow at the initiation of the extracorporeal treatment process or during the finishing phase while pumping saline solution through the fluid circuit 30. Implementation of these and similar alternatives will be described further below.

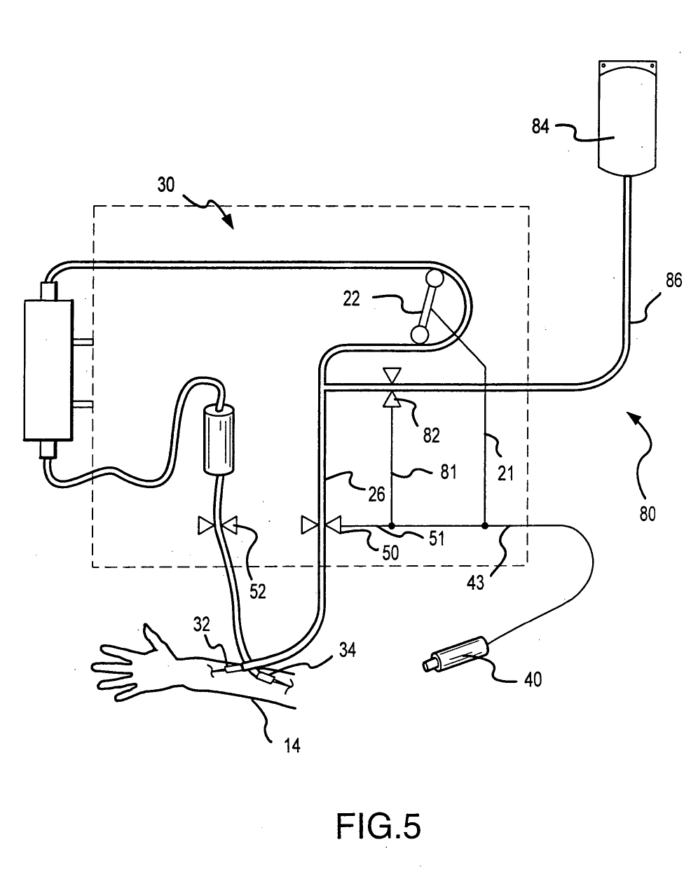

An alternative embodiment of the present invention is shown in FIG. 5. In particular, FIG. 5 is a schematic diagram much like that shown in FIG. 3 with the principal addition of a finishing treatment sub-assembly 80. Sub-assembly 80 generally includes a saline source such as the bag 84 shown which preferably contains a sterile physiological saline solution. Bag 84 is then connected to a tubing segment 86 which is also preferably operatively disposed relative to a tubing clamp 82 as will be described below. Segment 86 is then connected to the fluid circuit 30 at a point preferably on tubing segment 26 prior to pump 22; although it could be connected to any of the tubing segments at various points in the circuit 30. Note, in a preferred embodiment, the connection of saline line 86 occurs as close to the patient 14 as possible, or alternatively as close to clamp 50 as possible. As will be described, such a preferred close proximity to the patient 14 will provide for a desirable technique for reducing the quantity of blood left in the tubing circuit 30 at the completion of the blood treatment procedure.

As mentioned above, at the completion of a dialysis procedure, the pump 22, which had continually withdrawn blood from the patient 14, is shut off and the blood removal device 32 is removed from the vascular access site of the patient 14. The pump 22 may then be turned back on to flush the tubing circuit 30 with a saline solution which forces the blood remaining in the tubing circuit 30 through the processing unit (e.g., dialyzer) 24 and back into the patient 14 via the venous return device 34. Prior to this invention, it was customary to insert the arterial blood removal device 32 into a container of saline such as a saline bag to introduce the saline solution into the tubing circuit 30. However, this conventional saline insertion step and the pump re-starting step each required the operator to, at least momentarily and as described hereinabove, undesirably, divert attention away from the patient 14 and the patient's vascular access site.

To address this attention diversion dilemma, the remote control device 40 may be connected, as shown in the primary FIG. 5 alternative embodiment of the present invention, to the control unit 20 and fluid circuit 30 in a manner which allows for remote regulation of the saline component to be added to the fluid circuit 30. FIG. 5 shows the remote control device 40 schematically connected not only to the pump 22 but also to the arterial occlusion clamp 50 as well as to the saline solution occlusion clamp 82. These connections are shown schematically via the branches of internal control circuitry generally identified by the respective reference numerals 21, 51 and 81 as connected to the primary internal remote circuit connection 43 which was described generally before. Together, device 40 and occlusion clamps 50 and 82 provide for control of the flow of saline solution from the saline bag 84 through the saline tubing segment 86 into the fluid circuit 30. In particular, upon completion of the dialysis procedure, the operator may stop the blood pump 22 using the remote control device 40, remove the arterial blood removal device 32 from the patient 14, and begin the flow of saline solution for the finishing procedure by opening the saline occlusion clamp 82 using the remote control device 40. All of these steps may be completed without diverting attention away from the patient 14 or the patient's vascular access site. The closing of the arterial clamp 50 can proceed either before, simultaneously with or subsequent to the opening of the saline clamp 82 and is also preferably controlled via remote control device 40. A secondary alternative here may involve running pump 22 backwards for a brief period prior to closing clamp 50 and/or extricating removal device 32 from the patient. This may be performed after the opening of the saline clamp 82 and the introduction of saline solution into circuit 30. This would then provide for pushing blood in tubing segment 26 back into the patient 14 via removal device 32.

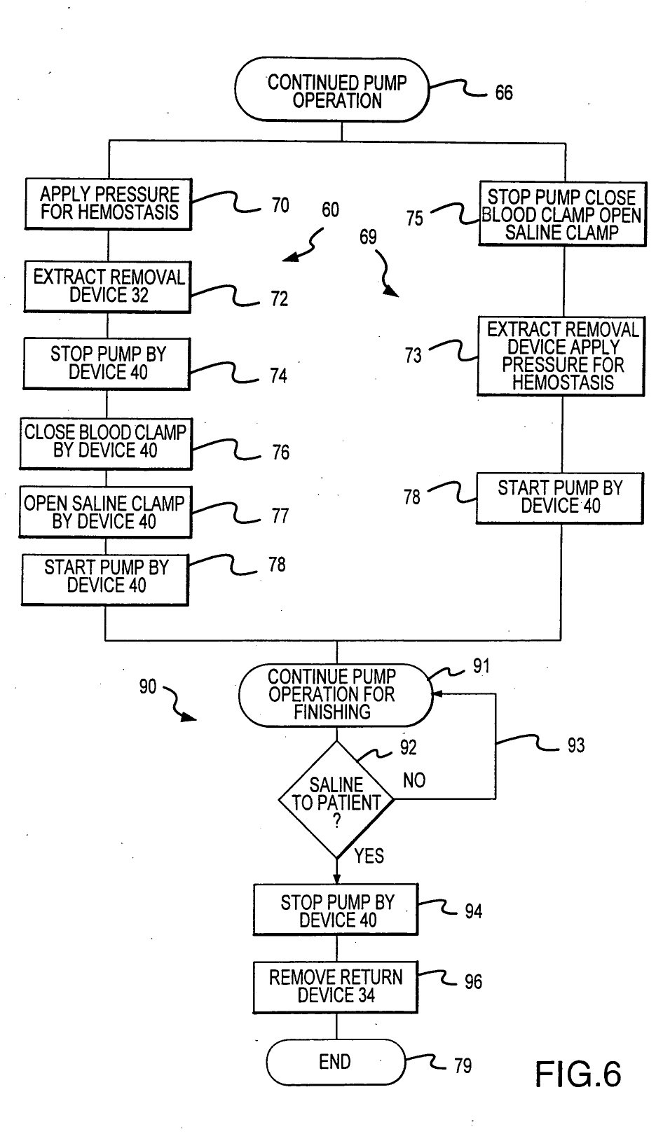

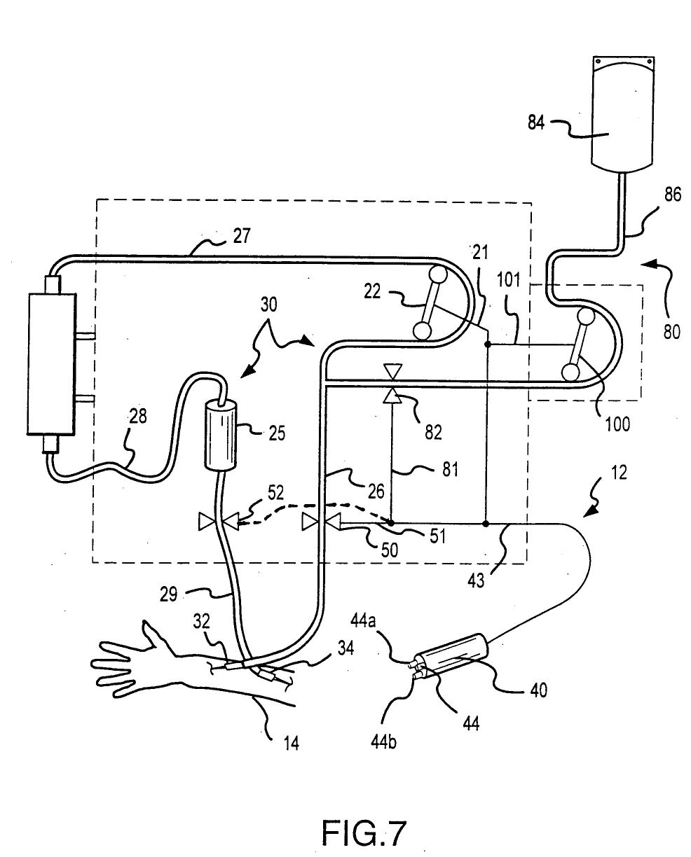

As a further aid in the understanding of the procedural benefit of using a remote control device 40 in the primary FIG. 5 finishing mode embodiment, reference is now made to the following descriptions relative to the process diagram of FIG. 6. As shown in FIG. 6, two optional paths are depicted after the continued pump operation process oval 66. These two options are generally identified by the respective reference numerals 68 and 69, respectively. Note, oval 66 represents the same general continued operating phase of extracorporeal processing as described at the end of the sub-process in FIG. 4A and/or at the beginning of the sub-process shown in FIG. 4B, above. First, optional path 68 shows a finishing procedure which is similar to that described for FIG. 4B, but is here used in accordance with the finishing sub-assembly 80 shown in FIG. 5. More particularly, the finishing steps of option 68 are similar to those set forth in FIG. 4B except for the addition of the two clamp control steps inserted after the pump stop step 74 as well as the addition of the operation finishing steps which are labeled as a group as flow path portion 90 of FIG. 6. As a consequence of these similarities, like numerals are used for like process boxes particularly in FIGS. 4B and 6, see particularly the hemostatic pressure box 70 and the device removal box 72. As to the clamp control steps, after the pump is stopped per step 74, then, according to the primary FIG. 5 alternative, the arterial clamp 50 is closed and the saline clamp 82 is opened as depicted by steps 76 and 77, respectively. Then, the pump 22 is started up again, per step 78, to pump saline solution from the saline bag 84 through saline tubing segment 86 into the fluid circuit 30. Multiple control buttons, switches, knobs or the like may be included on remote device 40 for control of the discrete machine components (clamp(s) and pump(s)) as desired; see the FIG. 7 description set forth below. Or, a single button 44 or the like which is capable of controlling more than one such component may be used. Options in this area are further described below.

Alternatively, as shown by optional path 69 in FIG. 6, the pump 22 may be stopped, the blood clamp 50 closed and the saline clamp 82 opened as shown by process box 75 prior to removal of the blood removal device 32 and hemostatic pressure application as these two sub-steps are depicted by process box 73. The pump 22 would then be re-started per box 78 preferably using remote control device 40 as before. One of the features of presenting this optional flow path 69 as discrete from option 68 is to illustrate the simultaneity and/or interchangeability of many of these steps in this initial part of the overall finishing process. In particular, as shown in step 75 the three sub-steps thereof are grouped together to demonstrate that they may be performed simultaneously with each other or in any relative order without adversely impacting the effectiveness of the overall process. This concept applies equally to the sub-steps of process box 73 as well. Moreover, a further distinction between options 68 and 69 is that in option 69, the patient care sub-steps of box 73 are shown after the primarily machine control sub-steps of box 75 whereas the corresponding machine control steps of option 68 (namely, steps 74, 76 and 77) were depicted after the patient care steps 70 and 72. This distinction is like the previous one in showing-that the overall process effectiveness is not vitally impacted by which steps occur first even if there may be other reasons why particular practitioners would prefer one sequence over another. Still further, the option 69 sequence having the machine steps performed first has another distinction in that by putting the machine control steps first; these steps can be performed at or by the machine without use of a device 40 prior to any practitioner attention necessarily being directed to the patient. Thus, instead of a multiple control device 40 capable of clamping and unclamping the blood and saline lines as well as controlling the pump 22 (as described above); device 40 need only be capable in this alternative of re-starting the pump 22 per box 78 after blood removal device extrication from the patient access site. Thus, by such a one feature (one button) operation, option 69 presents the possibility of greater user transparency in operation even with the added functionality of saline finishing as will now be described in more detail.

To complete the description of FIG. 6, the following are the details of flow portion 90 thereof. First, after the blood removal needle extrication step, the opening of the saline solution connection clamp 82, and the re-starting of pump 22 (per either box 78), then the pump 22 is operated continuously as represented by process oval 91 to force blood and saline solution forward through the fluid circuit 30. This operation is continued (per the negative loopback path line designated 93) until, as shown by the decision diamond 92, the saline has reached the patient. Once the practitioner notes that the saline has indeed reached the patient then the practitioner uses device 40 to stop the pump 22. This is shown by process box 94. Then, the practitioner devotes his or her attention to removing the return device 34. Hemostatic pressure is applied to the vascular return site in the same fashion as described above for the vascular blood removal access site, including applying hemostatic pressure (not shown). The procedure is now at an end, per oval 79, at least insofar as the overall extracorporeal procedure involves the remote control device 40 in this embodiment. An alternative additional step of closing the return clamp 52 could also be performed simultaneously or otherwise near in time to the pump stop step 94 described here. And, as before, this alternative functionality could be activated by proper manipulation of the remote control device 40.

Further, as noted above, a secondary alternative embodiment involving running pump 22 temporarily backwards (not directly shown in FIG. 6) may be performed by the movement of a few existing steps as well as the inclusion of a few additional steps at various points in the FIG. 6 flow chart. For example, the extrication of removal device 32 may be delayed until a certain amount of saline solution has been introduced into the fluid circuit 30. Then, the pump 22 may be run backwards to flush any blood in the tubing segment 26 back into the patient 14 via removal device 32. Then, the pump 22 may be re-stopped and the removal device 32 may be extracted from the patient. This alternative may then take the form in FIG. 6 of moving the hemostasis pressure and removal device extraction sub-steps 70/72 from option 68 or sub-step 73 from option 69 down to a point between sub-steps 91 and 92 or to a point even after sub-step 96. Thus, the open and close clamping sub-steps would remain as originally set and the saline pump operation would be run until the appropriate and/or a desired quantity of saline solution is pumped into the circuit 30. Then, the additional step of running the pump 22 backwards would be inserted before the continue pump operation step 91 with additional close blood clamp 50 and re-start pump 22, in the forward direction, steps (not shown) also added thereafter before finishing pump operation step 91.

Note, alternative single needle processing systems would make use of the present invention in a manner similar to the finishing procedures of either FIG. 4B (without a saline sub-assembly interconnection) or flow path portion 90 in FIG. 6 (with a saline connection) without substantial distinction over any of the herein described methods. As above, such single needle procedures could be viewed in FIG. 6, for example, as involving the movement (or elimination) of certain steps. Thus, the hemostasis pressure and removal device extraction sub-steps 70/72 from option 68 or sub-step 73 from option 69 would be moved down to a point immediately before or after or included within or in lieu of sub-step 96. Thus, the stop pump and open and close clamping sub-steps per either options 68 or 69 would remain as originally set and the saline pump operation would be run until the saline solution is pumped into and throughout the fluid circuit 30 until it reaches the patient 14 per steps 91 and 92. Then, the pump would be stopped per step 94 and the needle removal and hemostatic pressure application would be performed per step 96 or the combination of steps 70/72 or step 73 therewith. The conception of whether steps 70/72, or 73 are performed with or in lieu of (or before or after) step 96 mainly concerns what the single needle may be referred to as, whether as the removal, or the return needle or a combination of both. It is not truly a substantive distinction because only one needle is used, and its extrication would preferably be performed after the saline finishing procedure of steps 91 and 92 of FIG. 6. Similarly, the single needle of this example would be the equivalent of the removal device 32 of step 72 in FIG. 4B (without a finishing sub-assembly or process).

To aid in the operation of some of the previous embodiments, particularly that shown in option 68 of FIG. 6, the remote control device 40 may have a plurality of buttons, switches, knobs or other interactive control elements (not shown in FIG. 6, but see FIG. 7, below). In such an embodiment of the hand-held remote control device 40, a first button, such as button 44 as hereinabove described, could control the starting and stopping of the blood pump 22. A second button, switch, knob or the like could control the opening and closing of the occlusion clamp 50 (or clamp 52). And, a third button, switch, knob or the like could control the flow of saline solution via controlling the opening and closing of clamp 82. Multiple combinations and permutations are available such as adding a clamp 50 control to the FIG. 3 embodiment, or adding control(s) for both blood removal and return clamps 50 and 52 thereto. Still further, even more controls could be incorporated hereon for an optional additional pump 100 (see FIG. 7 for a description of one such optional pump, below) or other machine componentry. To prevent practitioner confusion, and maintain the invention objective of keeping the practitioner's attention directed toward the patient and the patient's vasculature, the plurality of buttons, switches, etc. could be different sizes, shapes, and/or colors, or could be arranged in particular pre-selected configurations. The practitioner would know which controlling button to press based upon where the controlling button was located on the device 40, or based upon the size or shape or color or other distinctive feature of the controlling button.

It is also possible that a single switching element such as button 44 or a like member could be provided which is capable of controlling a plurality of components such as the pump(s) and/or clamp(s) described above. One way to achieve this could be to use a multiple position switch, knob or the like in which its various discrete positions could be made to individually correspond to the operation of the discrete mechanical components. Another way could be based on having the multiple components all activated upon the single engagement (depression, switching, etc.) of a single button. For example, in the optional pathway 69 of FIG. 6, a single depression of a push-button 44 or the like could be made to activate all of the sub-steps of process box 75; namely, stop pump 22, close blood clamp 50, and open saline clamp 82. As described before, these sub-steps could be made to occur simultaneously, or in any preferred sequence. Such sequencing could then be programmable into the remote control sub-system 12 or into the control unit 20. Then, a subsequent engagement of the push-button 44, after the practitioner performed the appropriate patient care steps, could be made to merely restart pump 22 per box 78. And, a still further subsequent engagement of push-button 44 could be made to again stop pump 22 per box 94 of the pathway portion 90, described above. Also, as above, the optional closure of the return clamp 52 (not shown in FIG. 6), could similarly be tied to the singular operation of the push-button 44 coincidentally with the pump stop step 94. Again, this tying of steps could provide for simultaneous or sequential occurrences as desired; the sequence being programmable into either the remote control system 12 or the control unit 20. This programmability is foreseeably factory pre-programmed and/or user/practitioner programmable depending on various user desires or variables.

In yet another alternative, as shown in FIG. 7, the infusion of saline solution can be enhanced through use of an auxiliary pump 100. Pump 100 may be activated to pump additional fluid, preferably a sterile physiological saline solution, into the fluid circuit 30. Also, a branch 101 of internal line 43 may be used to connect the remote control device 40 to the additional pump 100 for remote regulation thereof. Pump 100 may be an integral part of control unit 20 or merely disposed adjacent thereto during operation. In use, an additional sub-step or a mere inclusion to a pre-existing sub-step could be envisioned in FIG. 6 to accommodate the functionality of this additional pump 100. In particular, as described above, both boxes 78 in FIG. 6 represent the starting of the pump 22 by the remote control device 40. However, these boxes 78 could also represent the starting of pump 100 as well. Thus, both pumps 22 and 100 could be substantially simultaneously or consecutively started by a single depression of the button 44 described throughout. Or, as shown in the FIG. 7 embodiment, multiple buttons could be disposed on the remote control device 40, and one such button, for example button 44 could be used for starting (and/or stopping) pump 22 and another button, for example button 44a could be used for starting (and/or stopping) pump 100. Other buttons could be used for other purposes as described above, such as a button 44b, which could be used for opening and/or closing the relevant tubing clamps, such as clamp 50, and/or clamp 82, for example.

Single needle operation with such a FIG. 7 embodiment would only involve the movement of the hemostatic pressure and needle extrication steps (see steps 70/72 or 73 in FIG. 6) to the end of the procedure as described above.

Also as introduced above, it is preferable to have the connection of the saline line 86 disposed as close as possible to clamp 50, while clamp 50 should be as close to the patient as possible. The reason for this in ordinary operation is that a minimum of blood should be left in the tubing set after the completion of a treatment because most patients in need of such treatments are sick and in need of as much of their own blood as possible. Thus, if the finishing process using a saline assembly 80 of either the FIG. 5 or the FIG. 7 embodiment involves only forward motion of pump 22 to push blood through the treatment device 24 prior to return to the patient, then some blood would be left behind in the tubing segment 26 between the removal device 32 and the closed tubing clamp 50, or even behind the connection point of the saline line 86 and the tubing segment 26. An alternative using the pump 100 shown in FIG. 7 could involve running the pump 100 when the main pump 22 is stopped (or possibly running backwards) and the clamp 50 is open and the removal device 32 is still inserted in the patient's access site. Thus, the blood in tubing segment 26 between the removal device 32 and the connection point of the saline line 86 and tubing segment 26 would be pushed backwards into the patient 14 followed by saline solution through needle 32. When the saline solution then reaches the patient via the removal needle 32, then the clamp 50 can be closed, the needle 32 removed, and pump 22 started with pump 100 remaining running (or temporarily stopped). Then, blood will be pushed forward, back to the patient 14 the other way, through processing device 24 and return needle 34 until saline reaches that needle per steps 91 and 92 of FIG. 6.

Still further, saline sub-assembly 80, with or without optional pump 100, may be used for saline infusion at any stage of treatment; whether during priming (see below), finishing (as an adjunct to the procedures set forth above), or also during blood processing as when a patient may have become dehydrated. In particular, should a patient become dehydrated during the dialysis procedure, immediate emergency rehydration may be started by opening the saline occlusion clamp 82, allowing saline solution to be introduced into the tubing circuit 30 so that it flows into the patient 14. The remote control device 40 of the present invention may be used to control this operation. Conventionally, when a patient would become dehydrated, the practitioner would have been required to manually locate a saline solution source and then-connect it to the tubing circuit 30 and then open the saline bag clamp (if applicable) to initiate flow for administration of liquid to the patient 14. This could also have required the operator to stop the blood pump 22, and perhaps close the arterial blood removal line clamp 50, all of these steps taking the practitioner's attention away from the patient in distress. With the present invention, the practitioner's attention may remain on the patient at all times, since these rehydrating steps can all be performed via remote control. Pump 100 as controlled by remote control 40 further enhances the infusion of saline solution to a dehydrated patient by allowing the practitioner to control the saline flow without removing attention from the patient. As mentioned above for pump 22, the speed of pump 100 may also be controllable from the remote control device 40. This may prove especially beneficial during rehydration procedures for quickness in rehydration and accuracy in volume control.

As mentioned, a remote device 40 can be used also for priming the fluid circuit prior to the extracorporeal treatment procedure. A system which may be used as an example is either of the FIG. 5 or 7 embodiments which each have a saline solution sub-assembly 80 connection to the fluid circuit 30. In either case, prior to connection to the patient 14, the saline solution can be fed into the circuit by the operation of pump 22, alone (FIG. 5) or in combination with pump 100. In either case, the pump 22 would preferably be run alternately in forward and reverse (or in reverse and then forward) so that saline solution can be forced into all tubing segments 26, 27, 28, and 29. Thus, if first run forward, pump 22 would be made to pump saline from saline bag 84 through tubing segment 86 into the upper part of segment 26 then into segment 27, into and through processing device 24 and into and through segment 28, air device 25 and segment 29 and ultimately to and through return device 34. Similarly, when run backwards, pump 22 would pump saline solution from bag 84 to and through the lower part of tubing segment 26 and then to and through the blood removal device 32. The starting and stopping and reverse operation of pump 22 can all be made controllable via remote device 40 in any of the manners described herein and/or implicitly equivalent hereto. Operation of clamps 50, 52 and/or 82 for fluid flow control during priming may also be automated via remote device 40. Also, the optional use of a pump 100 as an adjunct during priming could be made operable from device 40 in a manner similar to that described for the finishing procedure described above. Thus, pump 100 could be run in addition to or at times in lieu of pump 22 (as when priming the lower portion of segment 26 and removal device 32). After the fluid circuit 30 has been primed, then the blood removal and/or return devices may be inserted into the patient's access site(s) as described in relation to FIG. 4A above.

Priming using a remote device 40 may also be performed with a more conventional use of a separate saline bag connected to or near either the removal or return device 32 or 34. Thus, a bag connected thereto (such as either by pre-connection by another tubing segment, or by insertion of either device 32 or 34 thereinto, or otherwise as known in the art) can be used to prime the system by starting the pump 22 using remote device 40 and then stopping the pump 22 when the saline has appropriately reached and saturated all circuit elements in a fashion understood in the art. Then, process initiation procedures according to FIG. 4A can be started as described, including insertions of the access devices 32 and/or 34, or a single needle (not shown).

Several other embodiments of remote control devices according to the present invention are readily foreseeable within the scope and spirit hereof. For example, the remote control device may be foot activated by using a foot pedal instead of finger pressure. Similarly, a remote control device that need not be connected to the machine by a cable like cable 42 is further contemplated by this invention. Additionally, a device that recognizes oral commands given by the operator is also within the spirit and scope of the invention.

The physical adaptations of these three alternative embodiments, and others of like distinction, may be readily fashioned using understood concepts and elements without diminishing any aspect of the present invention. For example, a foot pedal remote control device would preferably be a device connected to the control unit 20 in a fashion such as is described above using a cable or the like such as cable 42 (see FIGS. 1-3, for example). Also, such a foot pedal device would preferably have a foot-activatable push-button or like switching member not unlike the push-button 44 described above, or any other alternative described therefor. The foot activatable push-button may, however, be larger and perhaps flatter than button 44 to simplify foot activation. Various foot buttons could additionally be incorporated onto the foot pedal device to encompass all the optional functionality described above, for operating clamps and pumps, among other elements, for example.

Similarly, remote control devices according to the present invention are not necessarily limited to hard wire or cable connections to the control unit 20. Remote control technologies using infrared or other electromagnetic wavelengths (e.g., optical, radio or micro waves) could also be used. These could be adapted into hand-held remote control devices such as device 40, or in foot or other bodily-activated devices as well. Ultrasound and/or audible sound activation such as through use of either an ultrasound and/or voice activation transducer are also readily adaptable herein. For example, the audible sound activation system may be a voice activation system, and may further be a voice recognition system. In any of these alternatives, the control unit 20 need only be adapted with a sound or electromagnetic receiver as these are known in other remote control arts, and adjusted to distinguish the intended incoming signals (sounds or electromagnetic waves) and with the proper electronic circuitry, convert the incoming signals into the proper corresponding controls for the pump(s) and/or clamp(s).

A further alternative involves the relative activation and deactivation of the remote control device. It is foreseeable that it may not be desirable to have the remote control permanently active, and thus a separate control element can be provided on the control unit 20 or on the remote device 40, itself, for activating and/or deactivating the remote control functionality. A simple push-button switch on either control unit 20 or device 40 may be provided for this purpose, or a sound or electromagnetic wave receiver similar to those described above, could be used for activating or deactivating the remote control function(s). Various security features can also be built into this activation/deactivation alternative. For example, it may be desirable to present a continuous or intermittent indication (such as a beeping sound or a flashing light) when the remote functionality has been activated (or alternatively, when deactivated). Such a r feature could be used to warn the practitioner that these important, perhaps life-impacting functions have been transferred from or activated in parallel with or in lieu of the controls on control unit 20 so that the practitioner can take the proper measure of care during the remote control use. Other security features could entail the use of special procedures for empowering the activation of the remote control functionality. Passwords, identification codes, sound matches (voiceprints, or other sound or voice recognition alternatives) or other like security checks may be required in order activate remote functionality. This will enable security from improper or accidental activation of potential life-endangering remote operations.

Once any of the needles described herein has been removed from the patient, it may be discarded in an understood manner according to the art; or, these needles may be safely secured for disposal according to a procedure and/or using an apparatus according to the invention described in the co-owned application filed on the same date contemporaneously herewith. This other invention is entitled NEEDLE HOLDING DEVICE by Jörgen Jönsson, a co-inventor of the present invention, application Ser. No. 09/737,444, now U.S. Pat. No. 6,511,416; and is incorporated herein by this reference. Thus, a needle 32 and/or 34 and/or a single needle (not shown) may be locked into a device according to this other invention and safely secured against accidental exposure or needle sticks, and then disposed of in a safe fashion.

Accordingly, a new and unique invention has been shown and described herein which achieves its purposes in an unexpected fashion. Numerous alternative embodiments readily foreseeable by the skilled artisan, though not explicitly described herein, are considered within the scope of the invention which is limited solely by the claims appended hereto.

1-60. (canceled)

61. An extracorporeal blood processing machine, directed to providing an operator a higher degree of simplification in controlling an extracorporeal fluid circuit comprising:
at least one tubing segment having a proximal end and a distal end;
the proximal end of the at least one tubing segment is proximal to and operatively engaged with a flow control device, which is operable to control the flow of fluid in and through the tubing segment;
the distal end of the at least one tubing segment is connected to at least one access device which is insertable into a patient's vascular access site for blood removal and/or return;
an operator controlled remote control sub-system for use in controlling the extracorporeal fluid circuit while the operator's attention is necessarily directed toward the patient and the patient's vascular access site;
the remote control subsystem comprising a remote control device having a switching member operably connected thereto;
the remote control device being disposed in operative communication with the flow control device such that activation of the switching member is effective to either initiate or halt the flow of fluid in the tubing segment;
and wherein the remote control sub-system is disjoined from the extracorporeal fluid circuit.

62. A remote control sub-system according to claim 1 in which the flow control device is a pump.

63. A remote control sub-system according to claim 2 wherein the control of the operation of said pump includes switching the pump into activated pumping operation.

64. A remote control sub-system according to claim 2 wherein the control of the operation of said pump includes switching the pump into deactivated pumping operation.

65. A remote control sub-system according to claim 1 in which the flow control device is a tubing clamp.

66. A remote control sub-system according to claim 5 wherein the control of the operation of the tubing clamp includes switching the tubing clamp into open, fluid flowing position.

67. A remote control sub-system according to claim 5 wherein the control of the operation of the tubing clamp includes switching the tubing clamp into closed, Do flow position.

68. A remote control sub-system according to claim 1 in which the remote control sub-system further comprises a cable having first and second ends;
the first end being operably connected to the flow control device;
the second end being operably connected to the remote control device and the switching member of the remote control device; and
the cable thereby connecting the remote control device in operative communication with the flow control device.

69. A remote control sub-system according to claim 1 in which the remote control sub-system further comprises an electromagnetic wave communication system
wherein the electromagnetic waves produced by the remote control device are received by an electromagnetic wave receiver, and are converted into the proper corresponding controls for the flow control device.

70. A remote control sub-system according to claim 1 which further comprises a sound activation system,
the sound activation system providing the operative connection of the remote control device to the flow control device.

71. A remote control sub-system according to claim 10 in which the sound activation system comprises a sound receiving apparatus disposed in the remote control device; and
the switching member comprises an electrical switching circuit element
which is adapted to convert the incoming signals into the proper corresponding controls for the flow control device.

72. A remote control sub-system according to claim 10 in which the remote control device is connected to the flow control device.

73. A remote control sub-system according to claim 10 in which the sound activation system is a voice activation system.

74. A remote control sub-system according to claim 13 in which the voice activation system comprises a voice recognition system.

75. A remote control sub-system according to claim 1 in which the flow control device is a pump and in which the at least one tubing segment of the fluid circuit is operatively engaged with a tubing clamp such that the tubing clamp is operable to control the flow of fluid in and through the tubing segment,
whereby the remote control device is further disposed in operative communication with the tubing clamp such that activation of the switching member is effective to control the operation of the tubing clamp.

76. A remote control sub-system according to claim 1 in which the flow control device is a pump and in which the fluid circuit has a second tubing segment operatively connected therein,
the second tubing segment being operatively engaged with a tubing clamp such that the tubing clamp is operable to control the flow of fluid in and through the second tubing segment
and whereby the remote control device is further disposed in operative communication with the tubing clamp such that activation of the switching member is effective to control the operation of the tubing clamp.

77. A remote control sub-system according to claim 1 in which the flow control device is a first pump and in which the fluid circuit has a second tubing segment operatively connected therein,
the second tubing segment being operatively engaged with a second pump such that the second pump is operable to control the flow of fluid in and through the second tubing segment,
whereby the remote control device is further disposed in operative communication with the second pump such that activation of the switching member is effective to control the operation of the second pump.

78. A remote control sub-system according to claim 17 whereby the second pump is adapted to be activated to pump an additional fluid into the fluid circuit.

79. A remote control sub-system according to claim 18 wherein the additional fluid is saline solution.

80. A remote control sub-system according to claim 1 in which the at least one tubing segment is also operatively engaged with a tubing clamp such that the tubing clamp is operable to control the flow of fluid in and through the tubing segment,
whereby the remote control device further comprises a second switching member which is disposed in operative communication with the tubing clamp and is thereby adapted to control the operation of the tubing clamp.

81. A remote control sub-system according to claim 15 in which the remote control device further comprises a second switching member which is disposed in operative communication with the tubing clamp and is thereby adapted to control the operation of the tubing clamp.

82. A remote control sub-system according to claim 16 in which the remote control device further comprises a second switching member which is disposed in operative communication with the tubing clamp and is thereby adapted to control the operation of the tubing clamp.

83. A remote control sub-system according to claim 17 in which the at least one tubing segment of the fluid circuit is operatively engaged with a tubing clamp such that the tubing clamp is operable to control the flow of fluid in and through the tubing segment, and
in which the remote control device further comprises a second switching member which is disposed in operative communication with the tubing clamp and is thereby adapted to control the operation of the tubing clamp.

84. A remote control sub-system according to claim 1 which further comprises a foot pedal remote control device.

85. A foot pedal remote control device according to claim 24 which has at least one foot-activatable push button or like switching member.

86. A remote control sub-system according to claim 1 which further comprises a hand-held remote control device.

87. A hand-held remote control device of claim 26 further comprising a thumb-engaging push button.

88. A disjoined remote control sub-system of claim 1 which may be operated from the patient's side adjacent to the vascular access blood removal and/or return site.
